(19) United States
(12) Patent Application Publication
(10) Pub. No.: US 2009/0163606 A1
(43) Pub. Date: Jun. 25, 2009

(54) ADDITIVES FOR CONTROLLED DISPERSION OF AQUEOUS SUSPENSIONS AND SUSPENSION COMPRISING SUCH ADDITIVES

(76) Inventors: GLEN HAROLD KIRBY, Liberty Township, OH (US); Brett Allen Boutwell, West Chester, OH (US)

Correspondence Address: GENERAL ELECTRIC COMPANY, GE AVIATION, ONE NEUMANN WAY MD H17, CINCINNATI, OH, 45215, US

(21) Appl. No.: 11/960,776

(22) Filed: Dec. 20, 2007

(51) Int. Cl. B01F 3/08; C07C 53/00

(52) U.S. Cl. 516/56; 516/9; 516/75; 516/59; 562/512; 562/590; 562/553; 562/104; 562/16

(57) ABSTRACT

Suspensions having additives for controlled dispersion including a solvent, an ion source, a particle source selected from a partially dissolving colloid or a non-dissolving colloid, and an additive where the additive is added to the solvent prior to the ion source and the particle source when the particle source is the partially dissolving colloid.

### STATEMENT OF GOVERNMENT RIGHTS

This invention was made, at least in part, with a grant from the Government of the United States (Contract No. N00019-04-C-0093, from the Department of the Navy). The Government may have certain rights to the invention.

### TECHNICAL FIELD

Embodiments described herein generally relate to additives for controlled dispersion of aqueous suspensions and suspensions comprising such additives. More particularly, embodiments herein generally describe additives for controlled dispersion of aqueous suspensions wherein the additive comprises at least one composition selected from the group consisting of low molecular weight zwitterionic organic species or organic species having at least one hydroxycarboxylic acid group, and suspensions comprising such additives.

### BACKGROUND OF THE INVENTION

Colloidal suspensions are used for a wide range of applications, including ceramic component manufacture, and paints and coatings. A colloidal suspension generally consists of solid particles (colloid) suspended in an aqueous solvent. In addition to the suspended colloid, the suspension may contain dissolved organic polymers (negative, positive, and charge-neutral), organic monomers, inorganic cations, and inorganic anions. The organic polymers and monomers may be intentionally dissolved into the aqueous suspension to function as a green strength binder, particle dispersant defoamer, drying aid, or viscosity modifier. The inorganic cations and anions may be present for two reasons. First, cations and anions may leach into the suspension due to the partial dissolution of the colloid itself, or some contaminant species added to the suspension. Second, inorganic salts may be intentionally added to the suspension. For example, in ceramic processing, inorganic salts may be added as sintering aids or for chemistry modification.

One possible side effect from dissolution of inorganic cations and anions into the suspensions is that the organic ions can promote flocculation of the colloid. This phenomenon can result in undesired rheological changes, such as increased suspension viscosity and shear thinning behavior, as well as inhomogeneous agglomerate formation due to high local concentration of ions upon initial dissolution from the colloid or added salt. Moreover, these issues can worsen with the addition of either large amounts of monovalent salts, such as potassium (K⁺), or small amounts of divalent or trivalent ions, such as barium (Ba²⁺), calcium (Ca²⁺), aluminum (Al³⁺), yttrium (Y³⁺), and sulfate (SO₃²⁻), depending on the ionic strength, [I], of the suspension. The ionic strength of a suspension scales by the following relationship, [I]˜cᵢ zᵢ², where c and z are the concentration and valence, respectively, of ion “i” dissolved in the suspension. Thus, the ionic strength is four times larger for divalent cations, and nine times larger for trivalent cations, over that of monovalent cations at the same concentration.

Currently, there are two approaches commonly used to control the dispersion of colloid particles in a suspension wherein the suspension contains dissolved anions and cations. The first approach generally involves using a dispersant having a comb-like architecture wherein the “backbone” of the comb is a polyelectrolyte such as polyacrylic acid, and the “teeth” of the comb comprise a charge-neutral, water-soluble polymer such as polyethylene oxide. See, for example, U.S. Pat. No. 7,053,125. This first approach can be effective if the source of the dissolved ions is the result of either an added salt or partial dissolution of the colloid particles. The second approach involves using a passivating agent, such as oxalic acid or phosphoric acid, to form a chemically inert layer on the surface of the suspended colloid particles. See, for example, U.S. Pat. No. 6,458,414. This second approach can be effective to block leaching but may not work if salt is intentionally added to the suspension.

Accordingly, there remains a need for additives that can control the dispersion of particles in suspensions containing anions and cations, regardless of how those ions came to be present.

### BRIEF DESCRIPTION OF THE INVENTION

Embodiments described herein generally relate to suspensions having additives for controlled dispersion comprising a solvent, an ion source, a particle source selected from the group consisting of a partially dissolving colloid or a non-dissolving colloid, and an additive wherein the additive is added to the solvent prior to the ion source and the particle source when the particle source comprises the partially dissolving colloid.

Embodiments herein also generally relate to suspensions having additives for controlled dispersion comprising a solvent, an ion source comprising a partially dissolving colloid, a particle source, and an additive wherein the additive is added to the solvent within about 24 hours after the ion source comprising the partially dissolved colloid.

Embodiments herein also generally relate to additives for controlling dispersion of aqueous suspensions comprising at least one composition selected from the group consisting of low molecular weight zwitterionic organic species or organic species comprising at least one hydroxycarboxylic acid group wherein the aqueous suspension comprises an ion source, and a particle source selected from the group consisting of a partially dissolving colloid or a non-dissolving colloid and wherein the additive is added to the solvent prior to the ion source and the particle source when the particle source comprises the partially dissolving colloid.

These and other features, aspects and advantages will become evident to those skilled in the art from the following disclosure.

### DETAILED DESCRIPTION OF THE INVENTION

Embodiments described herein generally relate to additives for controlled dispersion of aqueous suspensions and suspension comprising such additives.

As used herein, “additives” is used to refer to compositions selected from low molecular weight zwitterionic organic species (herein “zwitterionic species”) or organic species comprising at least one hydroxycarboxylic acid group (herein “organic species”). More specifically, the zwitterionic species can comprise polar molecules with both an ionizable anionic group (e.g., carboxylic acid group, sulfonic acid group, phosphonic acid group, etc), as well as a protonizable amine group within the same molecule. Such zwitterionic species may include, but should not be limited to, aminocarboxylic acids such as glycine and ethylenediaminetetraacetic acid (EDTA); amino-sulfonic acids such as 2-(N-morpholino)ethanesulfonic acid (MES), 3-(N-morpholino)propanesulfonic acid (MOPS), 4-(2-hydroxyethyl)-1-piperazineethanesulfonic acid (HEPES), piperazine-N,N′-bis(2-ethanesulfonic acid) (PIPES), and N-cyclohexyl-3-aminopropanesulfonic acid (CAPS); and aminophosphonic acids such as 1-aminoalkylphosphonic acid, and 2-(pyridylmethyl)phosphonic acid. Regarding the organic species, compositions in this group may include, but should not be limited to, citric acid, polycitric acid, gluconic acid, polygluconic acid, tartaric acid, malic acid, salicylic acid, hydroxysalicylic acid, and sugars such as glucose, dextrose, sucrose, and mannose.

Without intending to be limited by theory, it is believed that when included in a suspension containing ions, the previously described additives can form stable complexes with the ions in the suspension. This formation of stable complexes prevents the ions from participating in interactions that would otherwise result in particle flocculation, and the associated problems of increased suspension viscosity, shear thinning behavior, and inhomogeneous agglomerate formation.

There are numerous methods by which the previously described additives can be added to aqueous suspensions to achieve the desired stabilization effect, as explained herein below. However, regardless of the method used, in addition to the additive, the aqueous suspensions can comprise a solvent, an ion source, a particle source, and optional additional species including dispersants, binders, plasticizers, and defoamers.

As used herein, the “solvent” may comprise water, to which the ion source and additional species may be added. As will be explained herein below, the ion source and the additional species may be added to the solvent at different times during the making of the aqueous suspension.

As mentioned, at least one ion source may be included in the aqueous suspensions. As used herein, “ion source” refers to an inorganic salt, a dissolving colloid, a partially dissolving colloid, a contaminate, and a combination thereof.

As used herein, inorganic “salt” refers to any conventional inorganic salt known to those skilled in the art. In general, such salts can dissolve 100% when introduced into a solvent.

A dissolving colloid is a colloid that completely dissolves in the solvent. Since the dissolving colloid acts in a similar manner to the previously discussed salts, for purposes of the present embodiments, dissolving colloids are considered equivalent to salts. Compositions suitable for use as colloids are discussed herein below.

If a partially dissolving colloid is included as an ion source, the partially dissolving colloid can expel ions into the solvent, but remain a particle ranging in size from about 10 nm to about 10 μm upon completion of the dissolution process. In this way, partially dissolving colloids can serve as both an ion source and a particle source, as explained in greater detail below. Partially dissolving colloids may change in surface chemistry due to selective ion leaching away from the particle, or alternately, the particle could uniformly dissolve without any change in the surface chemistry. Moreover, dissolution of the partially dissolving colloid can continue until one of several occurrences takes place. In one embodiment, the dissolution process may stop because the diffusion of ions through the ion-depleted surface region of the partially dissolving colloid becomes a rate limiting step. In another embodiment, the dissolution process may stop because the surface of the partially dissolving colloid becomes passivated as a result of chemical changes in the solution, such as a change in pH. In another embodiment, the dissolution process may stop because the suspension becomes saturated with the expelled ions, thereby achieving a state of thermodynamic equilibrium.

As used herein, “contaminate” refers to any supply of incidental ions. Contaminates can originate from a variety of sources including, but not limited to contaminated water used as the solvent, the containers holding the slurry, the pipes through which the slurry is pumped during processing, accidental salt addition, and the like.

Regardless of whether the ion source comprises a salt, a dissolving colloid, a partially dissolving colloid, a contaminate, or a combination thereof, the ion source can dissolve when added to the solvent to produce an “ion.” Ions resulting from the dissolved ion source may include, but should not be limited to, H₃O⁺, NH₄⁺, Li⁺, Na⁺, K⁺, Rb⁺, Cs⁺, Fr⁺, Be²⁺, Mg²⁺, Ca²⁺, Sr²⁺, Ba²⁺, Ra²⁺, Sc³⁺, Y³⁺, La³⁺, Ce³⁺, Ce⁴⁺, Pr³⁺, Nd³⁻, Pm³⁺, Sm³⁺, Eu³⁺, Gd³⁺, Tb³⁺, Dy³⁺, Ho³⁺, Er³⁺, Tm³⁺, Yb³⁺, Lu³⁺, Al³⁺, Cr²⁺, Cr³⁺, Fe²⁺, Fe³⁺, Ti³⁺, Ti⁴⁺, Mn²⁺, Mn³⁺, Mn⁴⁺, Co²⁻, Co³⁺, Ni²⁺, Ni³⁻, Cu⁺, Cu²⁺, Cu³⁺, Zn²⁺, Ga³⁺, Ge²⁺, Ge⁴⁺, Se²⁺, Se⁴⁺, Zr²⁺, Zr⁴⁺, Nb³⁺, Nb⁵⁻, Rh³⁺, Pd²⁺, Ag⁺, Cd²⁺, In⁺, In²⁺, In³⁺, Sn²⁺, Sn⁴⁺, Sb³⁺, Sb⁵⁺, Hf²⁺, Hf⁴⁺, Ta³⁺, Ta⁵⁺, Ir³⁺, Au³⁺, Hg²⁺, Hg₂²⁺, Tl⁺, Tl³⁺, Pb²⁺, Pb⁴⁺, Bi³⁺, Po²⁺, Ac³⁺, Th²⁺, Th⁴⁺, U⁺, U²⁺, UO₂⁺, V²⁺, V³⁺, Np³⁺, Np⁴⁻, NpO⁺, Pu³⁺, Pu⁴⁺; OH⁻, F⁻, Cl⁻, Br⁻, I⁻, At⁻, SO₃²⁻, S₂O₃²⁻, HSO₄⁻, SO₄²⁻, HSO₃⁻, PO₄³⁻, HPO₄²⁻, H₂PO₄⁻, PO₃³⁻, NO₂⁻, NO₃⁻, CO₃²⁻, HCO₃⁻, HCO₂⁻, MoO₄²⁻, WO₄²⁻, TcO₄⁻, RuO₄⁻, ReO₄⁻, C₂H₃O₂⁻, C₂O₄²⁻, HC₂O₄⁻, HS⁻, Te²⁻, NH₂⁻, OCN⁻, SCN⁻, CN⁻, p³⁻, S²⁻, O₂²⁻, As³⁻, AsO₄³⁻, AsO₃³⁻, BO₃³⁻, BrO₃₋, BrO⁻, ClO₃⁻, ClO₄₋, ClO₂⁻, ClO⁻, CrO₄²⁻, Cr₂O₇²⁻, IO₃⁻, MnO₄⁻, and combinations thereof.

A particle source can also be included in the aqueous suspension of the present embodiments. “Particle source” refers to a solid particulate that can become suspended in the solvent. The particle source may include a partially dissolving colloid, a non-dissolving colloid, or a combination thereof. As previously discussed, because a partially dissolving colloid can expel ions into the solvent, but remain a particle ranging in size from about 10 nm to about 10 μm upon completion of the dissolution process, it can serve as a particle source as well as an ion source. A “non-dissolving colloid” is a colloid that does not dissolve and therefore, does not contribute any ions to the suspension. Non-dissolving colloids can range in size from about 10 nm to about 10 μm.

As defined herein, the term “colloid,” whether dissolving, partially dissolving, or non-dissolving, may be selected from the group consisting of a ceramic particle, a glass particle, a metal particle, a polymeric particle, or a semiconductor particle. In general, the colloid can account for from about 0.0001 vol % to about 60 vol % of the total volume of the suspension (i.e. suspension plus colloid volume).

As used herein, “ceramic particle” can include, but should not be limited to, SiC, Si₃N₄, AlN, Na₂O, Li₂O, K₂O, Ag₂O, Tl₂O, Cu₂O, BeO, MgO, CaO, SrO, BaO, NiO, CdO, CoO, MnO, CuO, TeO, ZnO, SnO, PbO, FeO, HgO, PdO, AgO, TiO, VO, Sc₂O₃, Y₂O₃, La₂O₃, Ce₂O₃, Pr₂O₃, Nd₂O₃, Pm₂O₃, Sm₂O₃, Eu₂O₃, Gd₂O₃, Tb₂O₃, Dy₂O₃, Ho₂O₃, Er₂O₃, Tm₂O₃, Yb₂O₃, Lu₂O₃, Cr₂O₃, Al₂O₃, Fe₂O₃, Bi₂O₃, Co₂O₃, Sb₂O₃, Ni₂O₃, Mn₂O₃, B₂O₃, In₂O₃, Ga₂O₃, Pb₂O₃, Tl₂O₃, As₂O₃, Rh₂O₃, Ti₂O₃, W₂O₃, V₂O₃, TiO₂, ZrO₂, HfO₂, ThO₂, CeO₂, CrO₂, UO₂, TeO₂, SeO₂, SiO₂, MnO₂, TcO₂, GeO₂, SnO₂, PbO₂, PuO₂, RuO₂, WO₂, VO₂, Sb₂O₅, As₂O₅, V₂O₅, Nb₂O₅, Ta₂O₅, P₂O₅, CrO₃, MoO₃, ReO₃, WO₃, TeO₃, SeO₃, UO₃, Fe₃O₄, Co₃O₄, Mn₂O₇, Re₂O₇, OsO₄, RuO₄ or combinations thereof. Combinations of such ceramic particles may comprise mixtures of oxides, such as, but not limited to RE₂Si₂O₇, RE₂SiO₅, REAl₃O₅, REGa₃O₅, REFe₃O₅, AETiO₃, AEAlO₂, AEAl₄O₇, AEAl₁₂O₁₉, AEZrO₃, AEHfO₃, AECeO₃, RE₂Zr₂O₇, RE₂Hf₂O₇, REPO₄, AE₃(PO₄)₂, AETa₂O₆, AENb₂O₆, RETaO₄, RENbO₄, AlPO₄, ZrSiO₄, HfSiO₄, and indium tin oxide, wherein RE is a rare earth element selected from scandium (Sc), yttrium (Y), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu), and AE is an alkaline earth element selected from magnesium (Mg), calcium (Ca), strontium (Sr), and barium (Ba).

As used herein, “glass particle” can include, but should not be limited to, amorphous particles (i.e. particles having no crystalline peaks observed in a powder x-ray diffraction scan) comprising silicon dioxide (SiO₂) along with any of Na₂O, Li₂O, K₂O, Ag₂O, Tl₂O, Cu₂O, BeO, MgO, CaO, SrO, BaO, NiO, CdO, CoO, MnO, CuO, TeO, ZnO, SnO, PbO, FeO, HgO, PdO, AgO, TiO, VO, Sc₂O₃, Y₂O₃, La₂O₃, Ce₂O₃, Pr₂O₃, Nd₂O₃, Pm₂O₃, Sm₂O₃, Eu₂O₃, Gd₂O₃, Tb₂O₃, Dy₂O₃, Ho₂O₃, Er₂O₃, Tm₂O₃, Yb₂O₃, Lu₂O₃, Cr₂O₃, Al₂O₃, Fe₂O₃, Bi₂O₃, Co₂O₃, Sb₂O₃, Ni₂O₃, Mn₂O₃, B₂O₃, In₂O₃, Ga₂O₃, Pb₂O₃, Tl₂O₃, As₂O₃, Rh₂O₃, Ti₂O₃, W₂O₃, V₂O₃, TiO₂, ZrO₂, HfO₃, ThO₂, CeO₂, CrO₂, UO₂, TeO₂, SeO₂, SiO₂, MnO₂, TcO₂, GeO₂, SnO₂, PbO₂, PuO₂, RuO₂, WO₂, VO₂, Sb₂O₅, As₂O₅, V₂O₅, Nb₂O₅, Ta₂O₅, P₂O₅, CrO₃, MoO₃, ReO₃, WO₃, TeO₃, SeO₃, UO₃, Fe₃O₄, Co₃O₄, Mn₂O₇, Re₂O₇, OsO₄, RuO₄, and mixtures thereof. Furthermore, the glass particle may comprise any of fluorine (F), chlorine (CL), bromine (Br), iodine (I), and mixtures thereof.

As used herein, “metal particle” can include, but should not be limited to silicon (Si), nickel (Ni), copper (Cu), ruthenium (Ru), rhodium (Rh), palladium (Pd), silver (Ag), rhenium (Re), platinum (Pt), gold (Au), iron (Fe), iridium (Ir), cobalt (Co), chromium (Cr), tungsten (W), tantalum (Ta), niobium (Nb), molybdenum (Mo), vanadium (V), titanium (Ti), zirconium (Zr), hafnium (Hf), alloys thereof, and mixtures thereof. For example, metal particle alloys may include nickel-chromium-aluminum-yttrium (NiCrAlY), cobalt-chromium-aluminum-yttrium (CoCrAlY), steels, aluminides, silicides, and the like.

As used herein, “polymeric particle” may include, but should not be limited to, polyvinyl alcohol, polyvinyl butyral, silicone, polyacrylic acid, polyethylene, polystyrene, and the like. Emulsions of such polymeric particles are also commonly known as latex.

As used herein, “semiconductor particle” can include, but should not be limited to, gallium arsenide (GaAs), silicon (Si), germanium (Ge), indium antimonide (InSb), gallium phosphide (GaP), gallium nitride (GaN), zinc sulfide (ZnS), cadmium telluride (CdTe), cadmium selenide (CdSe), zinc telluride (ZnTe), and zinc selenide (ZnSe).

Turning to the additional species that may be included in the aqueous suspension, the dispersant may comprise polyacrylic acid, polymethacrylic acid, sodium polyacrylates, sodium polymethacrylates, polyvinyl phosphoric acid, sulfonated naphthalene formaldehyde condensate, polyvinyl sulfonic acid, and combinations thereof, the binder may comprise polyvinyl alcohol, polyethylene oxide, xanthan gum, guar gum, methylcellulose, cellulose derivatives, and combinations thereof, the plasticizer may comprise glycerin, glycerol, and ethylene glycol, and the defoamer may comprise organic surface active agents, such as Surfynol® 502, and Surfynol® 420.

While the process for making the aqueous suspensions of the present embodiments can vary, in general, the additive may be added to the aqueous solvent either prior to the addition of the ion source, within 24 hours of the addition of the ion source, as explained herein below.

In one embodiment, the additive may be introduced before a dissolving colloid. In this example, the dissolving colloid acts as a “salt,” and therefore, as an ion source. In another embodiment, the additive can be introduced after a non-dissolving colloid, but before a salt. In another embodiment, the additive can be introduced before both of a non-dissolving colloid and a salt. In yet another embodiment, and as previously mentioned, the additive may be introduced up to about 24 hours after the addition of a partially dissolving colloid. After about 24 hours, most suspensions containing partially dissolved colloids will experience an increase in viscosity if an additive is not added. In still another embodiment, the additive can be introduced into a suspension containing a non-dissolving colloid (and no added salt) to prevent flocculation into a strong gel resulting from a contaminate, as defined herein.

Other processing parameters for consideration include stirring, and temperature. More particularly, it can be desirable to stir the suspension at least until the additive dissolves. However, stirring may be continued beyond the dissolution of the additive to maintain the homogeneity of the suspension and/or reduce the settling or breaking up a weak gel, if formed.

Regarding the temperature, it can be desirable to maintain the temperature of the aqueous suspension below about 100° C. (about 212° F.), and in one embodiment, below about 50° C. (about 122° F.), throughout the making thereof Allowing the temperature of the aqueous suspension to rise above about 100° C. may increase the solubility of certain colloids and may require more of the additive to achieve the desired stability. Moreover, at temperatures above about 100° C. the water used as the solvent will turn to steam.

The resulting aqueous suspension comprising additives for controlled dispersion can show reduced colloid flocculation in the presence of ions because the additives can promote the formation of stable complexes with the ions in the suspension. This formation of stable complexes prevents the ions from participating in the reactions that would otherwise result in particle flocculation, and the associated problems of increased suspension viscosity, shear thinning behavior, and inhomogeneous agglomerate formation.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A suspension having additives for controlled dispersion comprising:
a solvent;
an ion source;
a particle source selected from the group consisting of a partially dissolving colloid or a non-dissolving colloid; and
an additive
wherein the additive is added to the solvent prior to the ion source and the particle source when the particle source comprises the partially dissolving colloid.

2. The suspension of claim 1 wherein the solvent comprises water.

3. The suspension of claim 2 wherein the additive comprises at least one composition selected from the group consisting of low molecular weight zwitterionic organic species or organic species having at least one hydroxycarboxylic acid group.

4. The suspension of claim 3 wherein the low molecular weight zwitterionic organic species comprises a composition selected from the group consisting of aminocarboxylic acids, amino-sulfonic acids, or aminophosphonic acids.

5. The suspension of claim 3 wherein the organic species having at least one hydrocarboxylic acid group comprises citric acid, polycitric acid, gluconic acid, polygluconic acid, tartaric acid, malic acid, salicylic acid, hydroxysalicylic acid, or sugars.

6. The suspension of claim 3 wherein the ion source is selected from the group consisting of a salt, a dissolving colloid, a partially dissolving colloid, a contaminate, and combinations thereof.

7. The suspension of claim 6 wherein the ion source dissolves to produce an ion selected from the group consisting of H₃O⁺, NH₄⁺, Li⁺, Na⁺, K⁺, Rb⁺, Cs⁺, Fr⁺, Be²⁺, Mg²⁺, Ca²⁺, Sr²⁺, Ba²⁺, Ra²⁺, Sc³⁻, Y³⁺, La³⁺, Ce³⁺, Ce⁴⁺, Pr³⁺, Nd³⁺, Pm³⁺, Sm³⁺, Eu³⁺, Gd³⁻, Tb³⁺, Dy³⁺, Ho³⁺, Er³⁺, Tm³⁺, Yb³⁺, Lu³⁺, Al³⁺, Cr²⁺, Cr³⁺, Fe²⁺, Ti³⁺, Ti⁴⁺, Mn²⁺, Mn³⁺, Mn⁴⁺, Co²⁺, Co³⁺, Ni²⁻, Ni³⁺, Cu⁺, Cu²⁺, Cu³⁺, Zn²⁺, Ga³⁺, Ge²⁺, Ge⁴⁺, Se²⁺, Se⁴⁺, Zr²⁺, Zr⁴⁺, Nb³⁺, Nb⁵⁺, Rh³⁺, Pd²⁺, Ag⁺, Cd²⁺, In⁺, In²⁺, In³⁺, Sn²⁺, Sn⁴⁺, Sb³⁺, Sb⁵⁺, Hf²⁺, Hf⁴⁺, Ta³⁺, Ta⁵⁺, Ir³⁺, Au³⁺, Hg₂²⁺, Tl⁺, Tl³⁺, Pb²⁺, Pb³⁺, Bi⁴⁻, Po²⁺, Ac³⁺, Th²⁻, Th⁴⁻, U⁺, U²⁺, U³⁺, UO₂²⁺, V³⁺, Np³⁻, Np⁴⁻, NpO⁻, Pu³⁺, Pu⁴⁺; OH⁻, F⁻, Cl⁻, Br⁻, I⁻, At⁻, SO₃²⁻, S₂O₃²⁻, HSO₄, SO₄²⁻, HSO₃⁻, PO₄³⁻, HPO₃²⁻, H₂PO₄⁻, PO₃³⁻, NO₂⁻, NO₃⁻, CO₃²⁻, HCO₃⁻, HCO₂⁻, MoO₄²⁻, WO₄²⁻, TcO₄⁻, RuO₄⁻, ReO₄⁻, C₂H₃O₂₋, C₂O₄²⁻, HC₂O₄⁻, HS⁻, Te²⁻, NH₂⁻, OCN⁻, SCN⁻, CN⁻, P³⁻, S²⁻, O₂²⁻, As³⁻, AsO₄³⁻, AsO₃³⁻, BO₃³⁻, BrO₃⁻, BrO⁻, ClO₃⁻, ClO₄₋, ClO₂₋, ClO⁻, CrO₄²⁻, Cr₂O₇²⁻, IO₃⁻, MnO₄⁻, and combinations thereof

8. The suspension of claim 6 wherein the colloid is selected from the group consisting of a ceramic particle, a glass particle, a metal particle, a polymeric particle, or a semiconductor particle.

9. The suspension of claim 8 wherein the ceramic particle comprises a composition selected from the group consisting of SiC, Si₃N₄, AlN, Na₂O, Li₂O, K₂O, Ag₂O, Tl₂O, Cu₂O, BeO, MgO, CaO, SrO, BaO, NiO, CdO, CoO, MnO, CuO, TeO, ZnO, SnO, PbO, FeO, HgO, PdO, AgO, TiO, VO, Sc₂O₃, Y₂O₃, La₂O₃, Ce₂O₃, Pr₂O₃, Nd₂O₃, Pm₂O₃, Sm₂O₃, Eu₂O₃, Gd₂O₃, Tb₂O₃, Dy₂O₃, Ho₂O₃, Er₂O₃, Tm₂O₃, Yb₂O₃, Lu₂O₃, Cr₂O₃, Al₂O₃, Fe₂O₃, Bi₂O₃, Co₂O₃, Sb₂O₃, Ni₂O₃, B₂O₃, In₂O₃, Ga₂O₃, Pb₂O₃, Tl₂O₃, As₂O₃, Rh₂O₃, Ti₂O₃, W₂O₃, V₂O₃, TiO₂, ZrO₂, HfO₂, ThO₂, CeO₂, CrO₂, UO₂, TeO₂, SeO₂, SiO₂, MnO₂, TcO₂, GeO₂, SnO₂, PbO₂, PuO₂, RuO₂, WO₂, VO₂, Sb₂O₅, As₂O₅, V₂O₅, Nb₂O₅, Ta₂O₅, P₂O₅, CrO₃, MoO₃, ReO₃, WO₃, TeO₃, SeO₃, UO₃, Fe₃O₄, Co₃O₄, Mn₂O₇, Re₂O₇, OsO₄, RuO₄, the mixtures thereof.

10. The suspension of claim 8 wherein the glass particle is an amorphous particle comprising silicon dioxide in combination with a composition selected from the group consisting of Na₂O, Li₂O, K₂O, Ag₂O, Tl₂O, Cu₂O, BeO, MgO, CaO, SrO, BaO, NiO, CdO, CoO, MnO, CuO, TeO, ZnO, SnO, PbO, FeO, HgO, PdO, AgO, TiO, VO, Sc₂O₃, Y₂O₃, La₂O₃, Ce₂O₃, Pr₂O₃, Nd₂O₃, Pm₂O₃, Sm₂O₃, Eu₂O₃, Gd₂O₃, Tb₂O₃, Dy₂O₃, Ho₂O₃, Er₂O₃, Tm₂O₃, Yb₂O₃, Lu₂O₃, Cr₂O₃, Al₂O₃, Fe₂O₃, Bi₂O₃, Co₂O₃, Sb₂O₃, Ni₂O₃, Mn₂O₃, B₂O₃, In₂O₃, Ga₂O₃, Pb₂O₃, Tl₂O₃, As₂O₃, Rh₂O₃, Ti₂O₃, W₂O₃, V₂O₃, TiO₂, ZrO₂, HfO₂, ThO₂, CeO₂, CrO₂, UO₂, TeO₂, SeO₂, SiO₂, MnO₂, TcO₂, GeO₂, SnO₂, PbO₂, PuO₂, RuO₂, WO₂, VO₂, Sb₂O₅, As₂O₅, V₂O₅, Nb₂O₅, Ta₂O₅, P₂O₅, CrO₃, MoO₃, ReO₃, WO₃, TeO₃, SeO₃, UO₃, Fe₃O₄, Co₃O₄, Mn₂O₇, Re₂O₇, OsO₄, RuO₄, and mixtures thereof.

11. The suspension of claim 8 wherein the metal particle comprises a composition selected from the group consisting of silicon, nickel, copper, ruthenium, rhodium, palladium, silver, rhenium, platinum, gold, iron, iridium, cobalt, chromium, tungsten, tantalum, niobium, molybdenum, vanadium, titanium, zirconium, hafnium, alloys thereof, and mixtures thereof.

12. The suspension of claim 8 wherein the polymeric particle comprises a composition selected from the group consisting of polyvinyl alcohol, polyvinyl butyral, silicone, polyacrylic acid, polyethylene, or polystyrene.

13. The suspension of claim 8 wherein the semiconductor particle comprises a composition selected from the group consisting of gallium arsenide, silicon, germanium, indium antimonide, gallium phosphide, gallium nitride, zinc sulfide, cadmium telluride, cadmium selenide, zinc telluride, or zinc selenide.

14. The suspension of claim 8 further comprising any one or more of:
a dispersant selected from the group consisting of polyacrylic acid, polymethacrylic acid, sodium polyacrylates, sodium polymethacrylates, polyvinyl phosphoric acid, sulfonated naphthalene formaldehyde condensate, polyvinyl sulfonic acid, and combinations thereof;
a binder selected from the group consisting of polyvinyl alcohol, polyethylene oxide, xanthan gum, guar gum, methylcellulose, cellulose derivatives, and combinations thereof; and
a plasticizer selected from the group consisting of glycerin, glycerol, and ethylene glycol.

15. A suspension having additives for controlled dispersion comprising:
a solvent;
an ion source comprising a partially dissolving colloid;
a particle source; and
an additive
wherein the additive is added to the solvent within about 24 hours after the ion source comprising the partially dissolved colloid.

16. The suspension of claim 15 wherein the solvent comprises water.

17. The suspension of claim 15 wherein the additive comprises at least one composition selected from the group consisting of low molecular weight zwitterionic organic species or organic species having at least one hydroxycarboxylic acid group.

18. The suspension of claim 17 wherein the low molecular weight zwitterionic organic species comprises a composition selected from the group consisting of aminocarboxylic acids, amino-sulfonic acids, or aminophosphonic acids.

19. The suspension of claim 17 wherein the organic species having at least one hydrocarboxylic acid group comprises citric acid, polycitric acid, gluconic acid, polygluconic acid, tartaric acid, malic acid, salicylic acid, hydroxysalicylic acid, or sugars.

20. The suspension of claim 17 wherein the ion source dissolves to produce an ion selected from the group consisting of H₃O⁺, NH₄⁺, Li⁺, Na⁺, K⁺, Rb⁺, Cs⁺, Fr⁺, Be²⁺, Mg²⁺, Ca²⁺, Sr²⁺, Ba²⁺, Ra²⁺, Sc³⁻, Y³⁺, La³⁺, Ce³⁺, Ce⁴⁺, Pr³⁺, Nd³⁺, Pm³⁺, Pm³⁺, Sm³⁺, Eu³⁺, Gd³⁺, Tb³⁺, Dy³⁺, Ho³⁺, Er³⁺, Tm³⁺, Yb³⁺, Lu³⁺, Al³⁺, Cr²⁺, Cr³⁺, Fe²⁺, Fe³⁺, Ti³⁺, Ti⁴⁺, Mn²⁺, Mn³⁺, Mn⁴⁺, Co²⁺, Co³⁺, Ni²⁻, Ni³⁺, Cu⁺, Cu²⁺, Cu³⁺, Zn²⁺, Ga³⁺, Ge²⁺, Ge⁴⁺, Se²⁺, Se⁴⁺, Zr²⁺, Zr⁴⁺, Nb³⁺, Nb⁵⁺, Rh³⁺, Pd²⁺, Ag⁺, Cd²⁺, In⁺, In²⁺, In³⁺, Sn²⁺, Sn⁴⁺, Sb³⁺, Sb⁵⁺, Hf²⁺, Hf⁴⁺, Ta³⁺, Ta⁵⁺, Ir³⁺, Au³⁺, Hg²⁺, Hg₂²⁺, Tl⁺, Tl³⁺, Pb²⁺, Pb⁴⁺, Bi³⁺, Po²⁺, Ac³⁺, Th²⁻, Th⁴⁺, U⁺, U²⁺, U³⁺, UO₂²⁺, V²⁺, V³⁺, Np³⁻, NpO⁻, Pu³⁺, Pu⁴⁻; OH⁻, F⁻, Cl⁻, Br⁻, I⁻, At⁻, SO₃²⁻, S₂O₃²⁻, HSO₄⁻, SO₄²⁻, HSO₃⁻, PO₄³⁻, HPO₄²⁻, H₂PO₄⁻, PO₃³⁻, NO₂⁻, NO₃⁻, CO₃²⁻, HCO₃⁻, HCO₂⁻, MoO₄²⁻, WO₄²⁻, TcO₄⁻, RuO₄⁻, ReO₄⁻, ReO₄⁻, C₂H₃O₂⁻, C₂O₄²⁻, HC₂O₄⁻, HS⁻, Te²⁻, NH₂⁻, OCN⁻, SCN⁻, CN⁻, O³⁻, S²⁻, O₂²⁻, As³⁻, AsO₄³⁻, AsO₃³⁻, BO₃³⁻, BrO₃⁻, BrO⁻, ClO₃⁻, ClO₄⁻, ClO₂⁻, ClO⁻, CrO₄²⁻, Cr₂O₇²⁻, IO₃⁻, MnO₄⁻, and combinations thereof.

21. The suspension of claim 15 wherein the particle source is selected from the group consisting of the partially dissolving colloid or a non-dissolving colloid.

22. The suspension of claim 21 wherein the colloid is selected from the group consisting of a ceramic particle, a glass particle, a metal particle, a polymeric particle, or a semiconductor particle.

23. The suspension of claim 22 wherein the ceramic particle comprises a composition selected from the group consisting of SiC, Si₃N₄, AlN, Na₂O, Li₂O, K₂O, Ag₂O, Tl₂O, Cu₂O, BeO, MgO, CaO, SrO, BaO, NiO, CdO, CoO, MnO, CuO, TeO, ZnO, SnO, PbO, FeO, HgO, PdO, AgO, TiO, VO, Sc₂O₃, Y₂O₃, La₂O₃, Ce₂O₃, Pr₂O₃, Nd₂O₃, Pm₂O₃, Sm₂O₃, Eu₂O₃, Gd₂O₃, Tb₂O₃, Dy₂O₃, Ho₂O₃, Er₂O₃, Tm₂O₃, Yb₂O₃, Lu₂O₃, Cr₂O₃, Al₂O₃, Fe₂O₃, Bi₂O₃, Co₂O₃, Sb₂O₃, Ni₂O₃, Mn₂O₃, B₂O₃, In₂O₃, Ga₂O₃, Pb₂O₃, Tl₂O₃, As₂O₃, Rh₂O₃, Ti₂O₃, W₂O₃, V₂O₃, TiO₂, ZrO₂, HfO₂, ThO₂, CeO₂, CrO₂, UO₂, TeO₂, SeO₂, SiO₂, MnO₂, TcO₂, GeO₂, SnO₂, PbO₂, PuO₂, RuO₂, WO₂, VO₂, Sb₂O₅, As₂O₅, V₂O₅, Nb₂O₅, Ta₂O₅, P₂O₅, CrO₃, MoO₃, ReO₃, WO₃, TeO₃, SeO₃, UO₃, Fe₃O₄, Co₃O₄, Mn₂O₇, Re₂O₇, OsO₄, RuO₄, and mixtures thereof.

24. The suspension of claim 22 wherein the glass particle is an amorphous particle comprising silicon dioxide in combination with a composition selected from the group consisting of Na₂O, Li₂O, K₂O, Ag₂O, Tl₂O, Cu₂O, BeO, MgO, CaO, SrO, BaO, NiO, CdO, CoO, MnO, CuO, TeO, ZnO, SnO, PbO, FeO, HgO, PdO, AgO, TiO, VO, Sc₂O₃, Y₂O₃, La₂O₃, Ce₂O₃, Pr₂O₃, Nd₂O₃, Pm₂O₃, Sm₂O₃, Eu₂O₃, Gd₂O₃, Tb₂O₃, Dy₂O₃, Ho₂O₃, Er₂O₃, Tm₂O₃, Yb₂O₃, Lu₂O₃, Cr₂O₃, Al₂O₃, Fe₂O₃, Bi₂O₃, Co₂O₃, Sb₂O₃, Ni₂O₃, Mn₂O₃, B₂O₃, In₂O₃, Ga₂O₃, Pb₂O₃, Tl₂O₃, As₂O₃, Rh₂O₃, Ti₂O₃, W₂O₃, V₂O₃, TiO₂, ZrO₂, HfO₂, ThO₂, CeO₂, CrO₂, UO₂, TeO₂, SeO₂, SiO₂, MnO₂, TcO₂, GeO₂, SnO₂, PbO₂, PuO₂, RuO₂, WO₂, VO₂, Sb₂O₅, As₂O₅, V₂O₅, Nb₂O₅, Ta₂O₅, P₂O₅, CrO₃, MoO₃, ReO₃, WO₃, TeO₃, SeO₃, UO₃, Fe₃O₄, Co₃O₄, Mn₂O₇, Re₂O₇, OsO₄, RuO₄, and mixtures thereof.

25. The suspension of claim 22 wherein the metal particle comprises a composition selected from the group consisting of silicon, nickel, copper, ruthenium, rhodium, palladium, silver, rhenium, platinum, gold, iron, iridium, cobalt, chromium, tungsten, tantalum, niobium, molybdenum, vanadium, titanium, zirconium, hafnium, alloys thereof, and mixtures thereof.

26. The suspension of claim 22 wherein the polymeric particle comprises a composition selected from the group consisting of polyvinyl alcohol, polyvinyl butyral, silicone, polyacrylic acid, polyethylene, or polystyrene.

27. The suspension of claim 22 wherein the semiconductor particle comprises a composition selected from the group consisting of gallium arsenide, silicon, germanium, indium antimonide, gallium phosphide, gallium nitride, zinc sulfide, cadmium telluride, cadmium selenide, zinc telluride, or zinc selenide.

28. The suspension of claim 22 further comprising any one or more of:
a dispersant selected from the group consisting of polyacrylic acid, polymethacrylic acid, sodium polyacrylates, sodium polymethacrylates, polyvinyl phosphoric acid, sulfonated naphthalene formaldehyde condensate, polyvinyl sulfonic acid, and combinations thereof;
a binder selected from the group consisting of polyvinyl alcohol, polyethylene oxide, xanthan gum, guar gum, methylcellulose, cellulose derivatives, and combinations thereof; and
a plasticizer selected from the group consisting of glycerin, glycerol, and ethylene glycol.

29. An additive for controlling dispersion of aqueous suspensions comprising:
at least one composition selected from the group consisting of low molecular weight zwitterionic organic species or organic species comprising at least one hydroxycarboxylic acid group
wherein the aqueous suspension comprises an ion source, and a particle source selected from the group consisting of a partially dissolving colloid or a non-dissolving colloid and wherein the additive is added to the solvent prior to the ion source and the particle source when the particle source comprises the partially dissolving colloid.

30. The additive of claim 29 wherein the low molecular weight zwitterionic organic species comprises a composition selected from the group consisting of aminocarboxylic acids, amino-sulfonic acids, or aminophosphonic acids.

31. The additive of claim 29 wherein the organic species having at least one hydrocarboxylic acid group comprises citric acid, polycitric acid, gluconic acid, polygluconic acid, tartaric acid, malic acid, salicylic acid, hydroxysalicylic acid, or sugars.
